(19) United States
(12) Patent Application Publication
(10) Pub. No.: US 2025/0296767 A1
(43) Pub. Date: Sep. 25, 2025

(54) CARTRIDGE WITH LASER MARKING

(71) Applicant: The Procter & Gamble Company, Cincinnati, OH (US)

(72) Inventors: Rahul VYAS, Singapore (SG); Bhavesh RAJWANI, Singapore (SG)

(21) Appl. No.: 19/082,593

(22) Filed: Mar. 18, 2025

Related U.S. Application Data
(60) Provisional application No. 63/566,987, filed on Mar. 19, 2024.

(51) Int. Cl. B65D 85/84; A61L 9/12

(52) U.S. Cl. CPC B65D 85/84; A61L 9/12; A61L 2209/131; A61L 2209/133; B65D 2203/12

(57) ABSTRACT

A cartridge for a volatile composition dispenser is provided. The cartridge includes a reservoir containing a volatile composition in liquid form and having an opening, a membrane enclosing the opening of the reservoir, the membrane configured to allow evaporation of the volatile composition, and a sealing substrate enclosing the opening of the reservoir. The membrane includes one or more markings obtainable by laser marking.

### FIELD OF THE INVENTION

The present disclosure relates to the field of cartridges for volatile composition dispensers.

### BACKGROUND OF THE INVENTION

Continuous non-energised, membrane-based air freshening products are a consumer-friendly way to provide pleasant fragrances, and other air-enhancing products such as malodor reducing compounds, to an environment such as a room. Typically, these products are sold in the form of a cartridge comprising a perfume composition and a membrane through which the perfume composition may evaporate, where the cartridge is contained within a single-use plastic housing. The housing may include useful information for a consumer, for example reminding the consumer the type of fragrance that is emitted and an intended use for the air freshening product (e.g. living room or bathroom). This information prevents consumer confusion when they need to purchase a replacement product. The entire product is single-use, resulting in plastic waste each time a product is disposed.

In order to increase sustainability, it is desirable to provide such products with a refillable housing, into which a new cartridge may be placed once a previous cartridge has reached the end of its life. Since the lifetime of air freshening products is typically several weeks or months, a consumer will have disposed of any original packaging by the time they come to purchase a replacement product, and may have forgotten what type of product they purchased. This problem is especially the case for air freshening products that frequently exist as part of product lines with a large number of possible scent options. Therefore, in order to prevent consumer confusion and enable a consumer to repurchase the same product should they desire to do so, it is beneficial for the cartridge to be clearly marked with information for a consumer to easily know the identity of the product (e.g. scent and intended use). However, it is challenging to achieve this in a consumer-friendly way. For example, in cartridges of the type disclosed in WO2017/192638A1, it is desirable that a reservoir portion of the cartridge is formed from an unmarked transparent plastic to enable a consumer to easily visualise a remaining amount of liquid perfume composition, meaning that it is undesirable to mark this portion of the cartridge. It is challenging to print such information on the other part of the cartridge, since this is a membrane that will become saturated with perfume and other organic materials such as solvents, during use, dissolving or blurring any ink that is used to print information on the membrane.

There is also a continuous need for membranes having improved evaporative performance. Manufacturers of air freshening products typically have multiple products in a product line, with different perfume/fragrance options available for a consumer to choose. However, a challenge in preparing such products is ensuring that the different perfumes/fragrance options have similar evaporation rates such that each product has equivalent performance and longevity.

Therefore, there is a need for a cartridge that overcomes some or all of the problems discussed above.

### SUMMARY OF THE INVENTION

The current inventors have surprisingly found that using a laser to mark the surface of a membrane to form text or images solves some or all of the problems discussed above. The resulting marked portions of a membrane are stable against the presence of organic solvents and other components of a perfume composition, so the text or images do not smudge, blur or disappear when the membrane is wetted with perfume composition containing organic solvents.

Therefore, examples of the present invention provide the following.
1. A cartridge for a volatile composition dispenser, the cartridge comprising:
   a reservoir containing a volatile composition in liquid form and having an opening;
   a membrane enclosing the opening of the reservoir, the membrane configured to allow evaporation of the volatile composition; and
   a sealing substrate enclosing the opening of the reservoir,
   wherein the membrane comprises one or more markings obtainable by laser marking.
2. The cartridge according to clause 1, wherein the membrane comprises polyethylene having a molecular mass of from about 3.5 million to about 7.5 million amu.
3. The cartridge according to clause 1 or 2, wherein the membrane has a thickness of from about 0.1 mm to about 1 mm,
   optionally wherein the membrane has a thickness of from about 0.2 mm to about 0.4 mm.
4. The cartridge according to any one of the preceding clauses, wherein the membrane is formed from a single sheet of material.
5. The cartridge according to any one of the preceding clauses, wherein the markings comprise alterations to the membrane structure through from 0.1%, to 20% of a thickness of the membrane, optionally from 0.5%, to 15%, more optionally from 1% to 10%.
6. The cartridge according to clause 5, wherein the markings comprise alterations to the membrane structure through from 2% to 7% of a thickness of the membrane.
7. The cartridge according to clause 6, wherein the markings comprise alterations to the membrane structure through from 3% to 5% of a thickness of the membrane.
8. The cartridge according to any one of the preceding clauses, wherein the markings cover from 0.5% to 40% of an evaporative surface area of the membrane,
   optionally from 1% to 30%, more optionally from 4% to 20%, most optionally from 6% to 15%.
9. The cartridge according to clause 8, wherein the markings cover from 6% to 15% of an evaporative surface area of the membrane.
10. The cartridge according to any one of the preceding clauses, wherein the laser marking comprises laser charring, laser coloration or laser dark marking,
   optionally wherein the laser marking comprises laser charring.
11. The cartridge according to any one of the preceding clauses, wherein the markings comprise instructions to a user.
12. The cartridge according to any one of the preceding clauses, wherein the cartridge is configured to provide a higher evaporation rate of volatile composition as compared to a corresponding cartridge in which the membrane does not comprise one or more markings obtainable by laser marking,
   optionally wherein said higher evaporation rate of volatile composition is at least 5% higher when measured over 28 days, such as at least 10% higher.
13. A method of making a volatile composition cartridge, comprising:
(i) providing a membrane that has been marked with a laser; and
(ii) manufacturing a volatile composition cartridge that comprises the membrane, wherein the volatile composition cartridge comprises a volatile composition that is configured to evaporate through the membrane.
14. The method according to clause 13, wherein the membrane in step (i) has been marked with a laser using laser charring, laser coloration or laser dark marking,
   optionally wherein the membrane in step (i) has been marked with a laser using laser charring.

### BRIEF DESCRIPTION OF THE FIGURES

FIG. 1 shows an expanded view of a cartridge according to a configuration.

FIG. 2 shows a scan of a membrane that has been laser marked as described herein.

FIG. 3 is a graph showing weekly weight loss data for cartridges according to a configuration.

FIG. 4 is a graph showing cumulative weight loss data for cartridges according to a configuration.

### DETAILED DESCRIPTION OF THE INVENTION

An examples of the invention provides a cartridge for a volatile composition dispenser, the cartridge comprising:
a reservoir containing a volatile composition in liquid form and having an opening;
a membrane enclosing the opening of the reservoir, the membrane configured to allow evaporation of the volatile composition; and
a sealing substrate enclosing the opening of the reservoir,
wherein the membrane comprises one or more markings obtainable by laser marking.

### Laser Marking

The invention involves markings on a membrane that are obtainable by (e.g. may be obtained by, or are obtained by) laser marking. As used herein, laser marking is to be understood as a process of changing the appearance of the membrane using a laser. Typically, the laser marking does not involve substantial ablation of the surface of the membrane, and instead involves structural and/or chemical changes to the membrane, such as oxidation. In some configurations, the laser marking may be laser charring, laser coloration or laser dark marking. In certain configurations, the laser marking may be laser charring. Laser marking may be performed with any appropriate machine known to a person skilled in the art, such as a Four-heads visual Laser Marking Machine available from Tongtian Science and Technology Company, China.

Providing information to a consumer in the form of markings on the membrane of the cartridge, using laser marking, provides a number of benefits to the consumer. For example, where the cartridge is a cartridge for insertion into a reusable housing, the markings may include instructions to a user, such as instructions for placing the cartridge into the membrane. The markings may also include a description or indication as to the identity of the cartridge, e.g. the specific scent within a product line. This reminds the consumer the identity of the cartridge when they come to remove it from the reusable housing at the end of the cartridge's life, and this reminder makes it easier for a consumer to repurchase the same cartridge.

As discussed above, the marked portions of a membrane are stable against the presence of organic solvents and other components of a perfume composition, so the markings (which may comprise text or images intended to convey information to a user) do not smudge, blur or disappear when the membrane is wetted with perfume composition containing organic solvents. This represents an advantage as compared to printing on the membranes using inks and pigments, which would be dissolved by a perfume composition including organic solvents. In addition, inks and pigments may have poor biodegradability, causing environmental concerns that do not apply to the use of laser marking.

The current inventors have surprisingly found that controlled laser marking of a portion of the membrane does not detrimentally affect the properties of the membrane. In particular, the resulting laser marking does not cause leakage or sweating of a perfume composition through the membrane.

The changes to the membrane structure that occur when the surface is marked also advantageously increase the evaporation rate of volatile composition. This allows for a simple and accurate fine-tuning of membrane performance during manufacturing of an air freshening product. In this way, a single membrane may be purchased in bulk and used for products that contain perfume compositions with varying volatilities. During the production of each individual product in a product line (e.g. having different perfume/fragrance compositions with different volatilities), the evaporative properties of each membrane may be easily tuned using laser marking over a greater or lesser area, so that the evaporative properties of a membrane are optimised for the specific perfume composition contained in each product. This enables consistent evaporation rates to be achieved for products containing perfumes with different volatilities, providing a perfumer designing new products a greater freedom in the choice of perfumes. This may allow a manufacturer of air freshening products to provide new products having new perfume compounds that were previously difficult to incorporate into a membrane-based air freshening product whilst maintaining the required performance parameters. Therefore, in some configurations the cartridge of the invention is configured to provide a higher evaporation rate of volatile composition as compared to a corresponding cartridge in which the membrane does not comprise one or more markings obtainable by laser marking. The higher evaporation rate may be, for example, at least 5% higher when measured over 28 days, or at least 10% higher, as compared to an equivalent membrane that does not comprise laser markings. The evaporation rate may be measured by placing an activated cartridge in an upright position for 28 days in a room having volume 18.2 m³ at a temperature of 23° C.±2° C., a relative humidity of 60%±5%, atmospheric pressure, and an airflow of 250 cubic metres per hour. The weight loss over this time may be determined and is presumed to be due to evaporation of volatile composition. The weight loss may be determined as an average of three separate experiments.

The laser marking of the membrane, according to the invention, also avoids the need for a separate leaflet or insert containing information to the consumer. Such information may be provided directly on the membrane, reducing the amount of material required to manufacture the consumer product, and therefore, reducing the amount of material that is disposed of by the consumer.

The laser marking may alter the structure of the membrane, such as by oxidising or charring the membrane. Thus, the markings on the membrane may comprise alterations to the membrane structure through from 0.1% to 20% of a thickness of the membrane. In this context, alterations are to be understood as encompassing any changes to the membrane that are caused by the laser. In some configurations, the markings may comprise alterations to the membrane structure through from 0.5% to 15% of a thickness of the membrane, from 1% to 10%, from 2% to 7%, such as from 3% to 5% of a thickness of the membrane.

For the avoidance of doubt, where multiple ranges are provided for a variable disclosed herein, any end point from any range for that variable may be provided with any other end point from any other range for the same variable. Thus, the thickness of the alterations to the membrane structure discussed above may be from:
0.5% to 1%, 0.5% to 2%, 0.5% to 3%, 0.5% to 5%, 0.5% to 7%, 0.5% to 10%;
1% to 2%, 1% to 3%, 1% to 5%, 1% to 7%, 1% to 10%;
2% to 3%, 2% to 5%, 2% to 7%, 2% to 10%;
3% to 5%, 3% to 7%, 3% to 10%;
5% to 7%, 5% to 10%; or
7% to 10%.

The markings may cover any appropriate membrane area. In some configurations, the markings may cover from 0.5% to 40% of an evaporative surface area of the membrane, though a skilled person will appreciate that a greater or lesser area may be marked. In further configurations, the markings may cover from 1% to 30%, from 4% to 20%, such as from 6% to 15% of an evaporative surface area of the membrane. In this context, evaporative surface area is the area of the membrane from which volatile composition may evaporate. The evaporative surface area does not include parts of the membrane that cannot become impregnated with volatile composition or that form part of a seal area.

### Cartridge

The invention, in an example, provides a cartridge for a volatile composition dispenser.

The cartridge is typically a single-use disposable cartridge that contains a volatile composition for release to a surrounding environment, and once a cartridge is depleted of volatile composition it may be disposed of. The cartridge is typically for placing into a reusable housing, such that once the cartridge is depleted of volatile composition it may be removed from the housing and replaced by a new cartridge. The use of single-use cartridges with a reusable housing reduces the amount of material contributed to landfill as compared to products that are entirely single-use (i.e. where the housing is single-use), and also uses a lower volume of material (e.g. plastic) during the manufacturing process. Nevertheless, a person skilled in the art will appreciate that the benefits provided by the current invention may be obtained in a cartridge that is provided within a single-use housing. For example, the ability to tune the evaporation rate provided by a membrane using laser marking, as described herein, may be beneficial in the context of a disposable single-use volatile composition dispenser.

The cartridge comprises a reservoir, a membrane, and a sealing substrate. The reservoir contains the volatile composition and is enclosed by both the membrane and the sealing substrate, which may enclose the reservoir in any order. Thus, the membrane may enclose the sealing substrate, or the sealing substrate may enclose the membrane.

The cartridge may comprise an outer peripheral seal area, in which a peripheral portion of the reservoir and membrane may be sealed together. In configurations where the sealing substrate encloses the membrane, a peripheral portion of the sealing substrate may be sealed to a peripheral portion of the membrane at the outer peripheral seal area, i.e. a peripheral portion of each of the reservoir, membrane and sealing substrate may be sealed together at the outer peripheral seal area, provided that the sealing substrate may nevertheless be removed from the cartridge. This may be the case where the edges of the reservoir and membrane are coterminous. Alternatively, the edge of the reservoir may extend beyond the edge of the membrane, and the sealing substrate may be sealed directly to the reservoir at a peripheral region of the reservoir that is beyond the edge of the membrane. In configurations in which the membrane encloses the sealing substrate and the cartridge also comprises a rupture mechanism for rupturing the sealing substrate, the outer peripheral seal area may comprise only a peripheral portion of the reservoir and membrane. In such configurations, the cartridge may also comprise an inner peripheral seal area in which an inner peripheral portion of the reservoir is sealed to the sealing substrate. This may be achieved by providing the reservoir with an intermediate step at an inner peripheral portion (e.g. between the outer peripheral portion and a main body of the reservoir), where the sealing substrate may be sealed to the intermediate step of the reservoir.

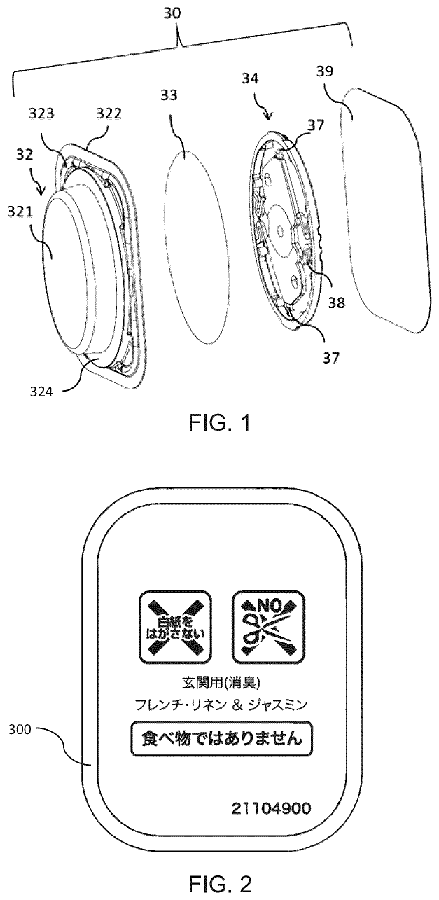

FIG. 1 shows an expanded view of a cartridge 30 according to a configuration. The cartridge 30 comprises a reservoir 32 having a main reservoir body 321, within which the volatile composition is stored. A sealing substrate 33 is sealably attached to and encloses the reservoir 32 at an inner peripheral seal area 324 to prevent the volatile composition from being released until the cartridge 30 is activated. Since the sealing substrate 33 in this configuration is internal relative to the membrane 39, it may be ruptured to release the volatile composition by actuating a rupture mechanism 34 positioned adjacent to the sealing substrate 33. One or more rupture elements 37 are arranged within the rupture mechanism 34 to puncture holes in the sealing substrate 33. The rupture element 37 may be a pin. The cartridge 30 may comprise a membrane 39 located on the exterior of the cartridge 30. The membrane 39 may be sealably attached to an outer peripheral seal area 322 located at a peripheral seal area 323 of the reservoir 32. The peripheral seal area 323 also comprises the inner peripheral seal area 324 in which the sealing substrate 33 is sealably attached to the reservoir 32. The membrane 39 may enclose the reservoir 32, the volatile composition, the sealing substrate 33, and the rupture mechanism 34. The membrane 39 may be configured to flex when a pressure or an actuation force is applied on the membrane 39. And the membrane 39 may be configured to porous so as to sustainably absorb and volatilize the volatile composition.

The reservoir, membrane, and sealing substrate are discussed in turn below.

### Reservoir

The reservoir may contain the volatile composition and has an opening that is enclosed by the membrane and sealing substrate.

The reservoir of the cartridge may typically be formed from a plastics material, which may advantageously be transparent to allow an easy view of a fill level of volatile composition within the reservoir. An example of a suitable material is polyethylene terephthalate (PET).

The reservoir may be configured for interfacing with a reusable housing. For example, the reservoir may be configured to be received by a window of a reusable housing, so that the cartridge is held securely within the housing when the housing is closed. The reservoir may therefore have a shape that is configured to correspond with a window of a reusable housing with which the cartridge is intended to be used, so the reservoir portion may be received and fit snugly within the window.

In such configurations, it may be advantageous for the reservoir to be formed from a transparent material, so that the fill level of volatile composition within the reservoir is visible from outside the reusable housing, such as through the window.

As mentioned above, the cartridge disclosed herein is typically a single-use cartridge for placing into a reusable housing. Thus, the cartridge typically does not comprise a housing of its own. In other words, the reservoir of the cartridge may be an outermost layer of the cartridge. In this context, “outermost” is to be understood as meaning that the cartridges do not include a substantial component outside the reservoir. For the avoidance of doubt, this does not exclude the presence of the membrane and sealing substrate enclosing an opening of the reservoir. In some configurations, the reservoir may nevertheless include a label or wrapping around the reservoir, which is intended to convey information to a user. However, the reservoir may be transparent as discussed herein, and in such cases the reservoir may typically not be covered by an additional label or wrapping so as to not obscure the reservoir.

### Membrane

The volatile composition is in liquid form and is configured to evaporate through a membrane. Accordingly, the cartridge comprises a microporous membrane, which for the sake of brevity may be referred to herein as “the membrane”. The membrane may enclose the reservoir such that volatile composition is unable to escape from the cartridge without passing through the membrane. The membrane may prevent the passage of liquid, such that the volatile composition is only able to escape the cartridge by evaporating through, or from, the membrane.

The membrane is vapor permeable and capable of wicking liquid, yet prevents free flow of liquid out of the membrane. Any suitable membrane may be used. Purely by way of example, certain properties that may result in advantageous membranes are discussed below. However, the invention is not limited to membranes having the properties below, and any membrane known in the art that allows the volatile composition to evaporate may be used in examples of the invention.

The membrane may have any appropriate volume average pore diameter, such as from 0.01 μm to 0.5 μm, such as from 0.02 μm to 0.3 μm, such as from 0.05 μm to 0.2 μm, more particularly from 0.065 μm to 0.15 μm since this may provide improvements with regard to evaporation rate and controlling leakage or sweating of volatile composition. In certain configurations, the membrane may have a volume average pore diameter of from 0.065 μm to 0.15 μm, from 0.07 to 0.12 μm, from 0.07 to 0.11 μm, or 0.08 to 0.1 μm.

In some configurations, the membrane may have a pore size distribution such that at least 50%, such as at least 60%, such as at least 70%, such as at least 80% or such as at least 90% of the pores of the membrane have a pore diameter of from 0.065 μm to 0.15 μm.

The membrane may comprise (e.g. be formed from) any appropriate material, such as a polymer. Suitable polymers include polyethylene, polypropylene, polyethylene/polypropylene copolymers, polytetrafluoroethylene, polyvinylidene difluoride, and polyacrylonitrile. A particular material that may be used is polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The membrane may have a thickness in the z-direction, of about 0.01 mm to about 1 mm, alternatively between about 0.2 mm to about 0.4 mm, from about 0.22 to about 0.37 mm, e.g. from about 0.25 to about 0.35 mm.

The membrane may be formed from a single piece, or single sheet, of material. In other words, the membrane may be not laminated. Thus, the membrane may be formed from a single sheet of polyethylene having a thickness as described above.

Those of ordinary skill in the art will appreciate that the surface area of the membrane can vary depending on the user preferred size of the cartridge. In some configurations, the (evaporative) surface area of the membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm². Particularly preferred membranes may have an evaporative surface area of from about 20 cm² to about 40 cm², such as from about 30 cm² to about 35 cm².

The membrane may form substantially all (e.g. at least 80%, at least 85%, at least 90% or at least 95%) of the surface area of a face of the cartridge. This advantageously allows the membrane to have a maximised evaporative surface area for the size of the cartridge, leading to improved release of volatile composition.

Thus, in some configurations the membrane may have an evaporative surface area of from about 20 cm² to about 40 cm², such as from about 30 cm² to about 35 cm² and form substantially an entire face of the cartridge.

In some configurations, the membrane may have an evaporative surface area of from about 20 cm² to about 40 cm², such as from about 30 cm² to about 35 cm² and the cartridge may have a maximum dimension of less than 10 cm, preferably less than 9 cm. This advantageously means that the cartridge has a compact size whilst retaining a high evaporative surface area. In such configurations, the membrane has a high size relative to the overall size of the cartridge, providing a high evaporation rate of volatile composition.

The membrane may have any appropriate porosity. For example, the membrane may have a porosity of from 45% to 70%, on a volume basis, such as from 45% to 65%. In certain configurations, the porosity may be from 50 to 70%, such as 55 to 65%.

The membrane may have any appropriate total pore volume, such as from 0.6 to 2 cm³/g. Typically, the total pore volume may be from 0.65 to 1.6 cm³/g, such as 0.7 to 1.5 cm³/g. In certain configurations, the total pore volume may be from 0.8 to 1.4 cm³/g.

The membrane may have any appropriate bulk density, such as from 0.3 to 0.8 g/cm³. Typically, the bulk density may be from 0.35 to 0.75 g/cm³, such as from 0.4 to 0.7 g/cm³. In certain configurations, the bulk density may be from 0.4 to 0.6 g/cm³.

Suitable membranes for the present invention include polyethylene membranes having the properties described herein, available from Microporous, LLC.

The membrane may comprise any suitable filler and plasticizer known in the art. Fillers may include finely divided silica, clays, zeolites, carbonates, and mixtures thereof. In one configuration, the membrane may be filled with about 30% to about 80%, by total weight, of silica.

In one aspect of the invention, the membrane may include a dye that is sensitive to the amount of volatile composition it is in contact with to indicate end-of-life. Alternatively, the membrane may change to transparent when in contact with a fragrance or volatile composition to indicate diffusion is occurring. Other means for indicating end-of-life that are known in the art are contemplated for the present invention.

### Sealing Substrate

The cartridge comprises a sealing substrate that encloses the reservoir, and hence, encloses the volatile composition. This prevents evaporation of the volatile composition for as long as the sealing substrate is in place and intact. The sealing substrate may be removed or ruptured to allow the volatile composition to evaporate. This removal or rupturing may be referred to herein as “activating” or “activation of” the cartridge. The cartridge is configured to be activated before use, i.e. the sealing substrate is configured to be ruptured or removed before use. In this context, “use” refers to enabling the volatile composition to evaporate from the cartridge, i.e. perform its function of dispensing the volatile composition by evaporation. In other words, rupturing or removing the sealing substrate enables evaporation of the at least one liquid volatile composition from the cartridge.

Non-limiting examples of suitable sealing substrates include an impermeable film, foil, or laminate, such as a flexible (e.g. polymeric) film, a flexible (e.g. metal) foil, or a composite material (e.g. a foil/polymeric film laminate). The impermeable film, foil or laminate is provided adhered to the cartridge to prevent evaporation of volatile composition. A particular example of a suitable sealing substrate is aluminium foil.

The sealing substrate may have any appropriate thickness, such as from 10 μm to 1 mm, from 15 μm to 100 μm, from 18 μm to 50 μm, or from 20 μm to 35 μm.

As mentioned above, there are two possible configurations for the order of the sealing substrate and the membrane.

In some configurations, the sealing substrate may be positioned between the membrane and the volatile composition, preventing the volatile composition from contacting the membrane. In such configurations, the cartridge may comprise a rupture mechanism. Thus, the cartridge may be configured for use with a housing that comprises rib elements that, upon closure of the housing, actuate the rupture mechanism, causing rupture of the sealing substrate and allowing volatile composition to pass through the ruptured sealing substrate and come into contact with the membrane. The volatile composition may then impregnate the membrane, from which it may evaporate. Suitable rupture mechanisms are described in detail in U.S. Pat. Nos. 10,561,754, 10,561,755 and 10,561,756.

In such configurations, the membrane is typically an outermost layer of the cartridge, and the presence of markings on the membrane may advantageously convey information to a consumer throughout the lifetime of the product.

In other configurations, the membrane may be positioned between the volatile composition and the sealing substrate, such that the volatile composition is in contact with the membrane but cannot evaporate and leave the cartridge because the membrane is enclosed by the sealing substrate. In such configurations, the cartridge may also be configured for use with a housing that, like the foregoing, comprises ribs which directly rupture the sealing substrate. However, this may result in only small holes in the sealing substrate, limiting the rate at which the volatile composition may evaporate from the cartridge. Therefore, in such configurations, the sealing substrate may be provided as a removable component (such as a removable film, foil or laminate) that encloses the membrane and prevents evaporation of the volatile composition before the sealing substrate is removed. When a sealing substrate of this type is removed from the cartridge, the entire membrane may be exposed to a surrounding environment, enabling an increased evaporation rate of volatile composition. In such cases, the sealing substrate may be provided with a pull tab to allow for easy removal of the sealing substrate from cartridge prior to use by a consumer.

In such configurations, once the sealing substrate has been removed the membrane will typically be an outermost layer of the cartridge, and the presence of markings on the membrane may advantageously convey information to a consumer once the sealing substrate has been removed. For example, the markings may identify the type of cartridge (e.g. the scent) so that a user is reminded the identity of the product at the end of life and is able to easily repurchase the same cartridge.

Where the sealing substrate is provided with a pull tab to facilitate removal of the sealing substrate, the pull tab may be any suitable size. For example, the pull tab may have a length of from about 20 mm or less, preferably about 15 mm or less, or more preferably about 10 mm or less. As another example, the pull tab may have a length of from about 2 mm to about 20 mm, preferably from about 5 mm to about 15 mm, or more preferably from about 5 mm to about 10 mm, specifically including all values within these ranges and any ranges created thereby. Similarly, the width of the pull tab may be about 20 mm or less, preferably about 15 mm or less or more preferably about 10 mm or less. As another example, the pull tab may have a width of from about 2 mm to about 20 mm, preferably from about 5 mm to about 15 mm, or more preferably from about 5 mm to about 10 mm, specifically including all values within these ranges and any ranges created thereby. The pull tab may be positioned in any suitable location on the sealing substrate. Preferably the pull tab is disposed adjacent the seal area.

Where the length and/or width of the pull tab exceed the above, the cartridges may be difficult to package. For example, the housing and the cartridge may be packaged together or separately. Where the cartridge is disposed within the housing, the housing may be in the open position or the closed position. When in the closed position, it would be desirable to ensure the pull tab is similarly disposed within the housing as well. Where the cartridge is disposed outside of the housing, it may be useful to show a portion of the pull tab to a consumer to demonstrate the facility with which the cartridges may be activated; however, where the length and/or width exceed the above values, it may be difficult to show only a portion while hiding the remainder of the pull tab.

As described, in some configurations, the sealing substrate may be a rupturable substrate. In other configurations, the sealing substrate may be a removable sealing substrate.

### Manufacturing

The invention, in an example, also provides a method of making a volatile composition cartridge, comprising:
(i) providing a membrane that has been marked with a laser; and
(ii) manufacturing a volatile composition cartridge that comprises the membrane, wherein the volatile composition cartridge comprises a volatile composition that is configured to evaporate through the membrane.

In some configurations, the membrane of step (i) has been marked with laser charring, laser coloration or laser dark marking. In some configurations, the method comprises a preliminary step of marking the membrane with a laser, such as by laser charring, laser coloration or laser dark marking. The cartridge, and particularly the membrane, may have any of the properties discussed hereinabove.

### EXAMPLES

UHMWPE membranes were obtained from PPG Industries, Inc and had the following properties.

| | |
|---|---|
| Bulk Density (g/ml) | 0.78 |
| Total Pore Volume (ml/g) | 0.53 |
| Volume Average Pore Diameter (μm) | 0.035 |
| Porosity (%) | 44 |
| Thickness (μm) | 280 |

The membranes were laser marked using a Four-heads visual Laser Marking Machine available from Tongtian Science and Technology Company, China with the settings listed in Table 1 below.

All samples were conducted using a laser power of 5 W and involved laser marking over approximately 9% of the membrane evaporative surface area.

TABLE 1
Membrane marking parameters

| | Laser | | | Pulse | Membrane | Cycle | |
|---|---|---|---|---|---|---|---|
| | Speed | Current | Frequency | Width | Temperature | Time | Marking |
| Example | (mm/s) | (A) | (kHz) | (μs) | after marking | (s) | Visibility |
| 1 | 4000 | 1 | 30 | 24.5 | Normal | 1.57 | Poor |
| 2 | 4000 | 1 | 30 | 22 | Normal | 1.57 | Medium |
| 3 | 4000 | 1 | 30 | 8 | Normal | 1.57 | High |
| 4 | 170 | 1 | 30 | 1 | Warm | 15.24 | High |
| 5 | 120 | 1 | 30 | 1 | Warm | 27.45 | Full |
| | | | | | | | thickness |
| | | | | | | | burn |
| 6 | 4000 | 1 | 30 | 13 | Not tested | 1.57 | High |
| 7 | 4000 | 1 | 30 | 17 | Not tested | 1.57 | High |

Example 5 caused full thickness burns, resulting in membrane failure. However, the laser settings used in these Examples may nevertheless be appropriate for thicker or more durable (e.g. denser) membranes, which may incur only partial-thickness charring under these settings.

Examples 2 and 3 had a short laser marking cycle time and provided good marking visibility. These Examples were therefore selected for further analysis as the short cycle time enables high-throughput manufacturing and the good marking visibility ensures high consumer satisfaction. Examples 6 and 7 also provide similar cycle times and good marking visibility, but were not assessed further as their properties are believed to be between those of Examples 2 and 3.

Examples 2 and 3 were analysed under a microscope to assess the marking (charring) thickness. Example 2 involved a marking thickness of 10.2 μm (3.7% of the membrane thickness), while Example 3 involved a marking thickness of 12 μm (4.4% of the membrane thickness). FIG. 2 shows the membrane of Example 2 with markings produced using laser marking. The border region 300 represents a seal region in which the membrane is sealed to the reservoir of the cartridge. The edges of the border region 300 are shown for illustrative purposes and were not generated with laser marking. The images and text at the inner, evaporative, portion of the membrane were generated by laser marking.

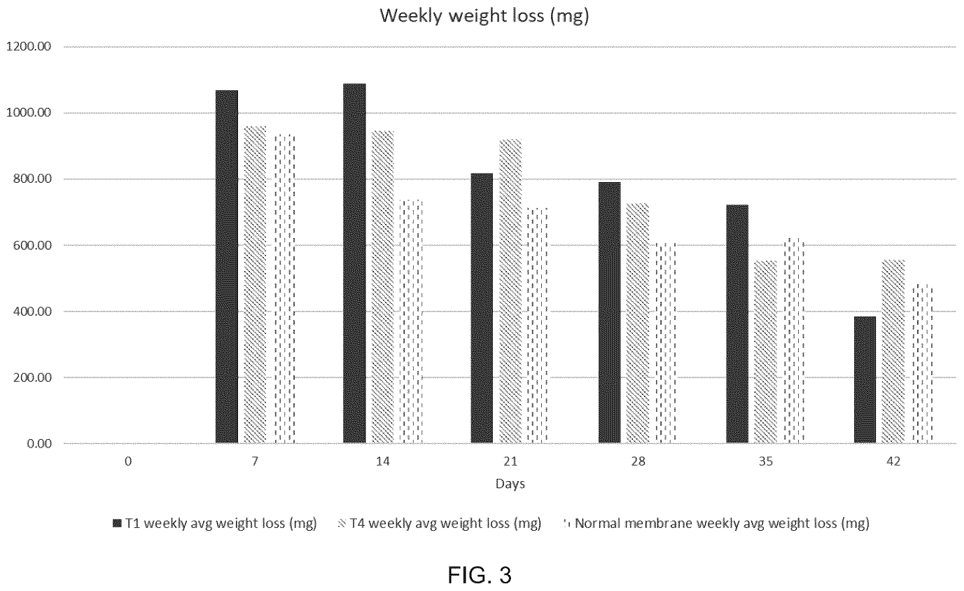

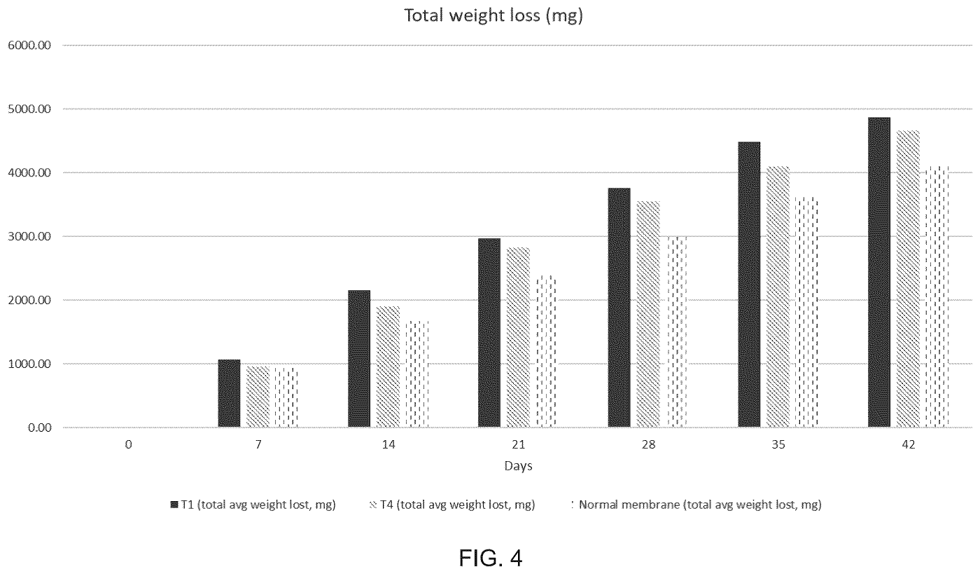

Each of Examples 2 and 3 were tested for the release of volatile composition. Three cartridges having membranes according to each of Examples 2 and 3 were tested for weight loss over 6 weeks (42) days in a room having volume 18.2 m³ at a temperature of 23° C.±2° C., with a relative humidity of 60%±5%, atmospheric pressure, and an airflow of 250 cubic metres per hour. The cartridges contained a volatile composition present in Febreze® Deodorant for Entrance, French Linen and Jasmine scent, as marketed in Japan in January 2024 by The Procter & Gamble Company. The weight loss corresponds to evaporation of volatile composition, and a greater weight loss indicates greater evaporation. Average weight loss results for the three samples are shown in FIG. 3 (weekly weight loss) and FIG. 4 (cumulative weight loss), where Example 2 is referred to as “T4”, and Example 3 is referred to as “T1”. Surprisingly, both Examples 2 and 3 provided improved release of volatile composition as compared to the unmarked membrane, confirming the ability of laser marking as discussed herein to tune and improve the evaporative properties of a membrane. These improvements in release of volatile composition were obtained without any leakage or sweating of the volatile composition through the membrane.

As used in this specification and the appended claims, the singular forms “a”, “an”, and “the” include plural references unless the content clearly dictates otherwise. Thus, for example, “a volatile material” may include more than one volatile material. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular configurations of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cartridge for a volatile composition dispenser, the cartridge comprising:
a reservoir containing a volatile composition in liquid form and having an opening;
a membrane enclosing the opening of the reservoir, the membrane configured to allow evaporation of the volatile composition; and
a sealing substrate enclosing the opening of the reservoir,
wherein the membrane comprises one or more markings obtainable by laser marking.

2. The cartridge according to claim 1, wherein the membrane comprises polyethylene having a molecular mass of from about 3.5 million to about 7.5 million amu.

3. The cartridge according to claim 1, wherein the membrane has a thickness of from about 0.1 mm to about 1 mm.

4. The cartridge according to claim 3, wherein the thickness is from about 0.2 mm to about 0.4 mm.

5. The cartridge according to claim 1, wherein the membrane is formed from a single sheet of material.

6. The cartridge according to claim 1, wherein the markings comprise alterations to the membrane structure through from 0.1%, to 20% of a thickness of the membrane.

7. The cartridge according to claim 1, wherein the markings comprise alterations to the membrane structure through from from 0.5%, to 15% of a thickness of the membrane.

8. The cartridge according to claim 1, wherein the markings comprise alterations to the membrane structure through from 2% to 7% of a thickness of the membrane.

9. The cartridge according to claim 1, wherein the markings comprise alterations to the membrane structure through from 3% to 5% of a thickness of the membrane.

10. The cartridge according to claim 1, wherein the markings cover from 0.5% to 40% of an evaporative surface area of the membrane.

11. The cartridge according to claim 1, wherein from 4% to 20% of an evaporative surface area of the membrane.

12. The cartridge according to claim 11, wherein the markings cover from 6% to 15% of an evaporative surface area of the membrane.

13. The cartridge according to claim 1, wherein the laser marking comprises laser charring, laser coloration, or laser dark marking.

14. The cartridge according to claim 1, wherein the laser marking comprises laser charring.

15. The cartridge according to claim 1, wherein the markings comprise instructions to a user.

16. The cartridge according to claim 1, wherein the cartridge is configured to provide a higher evaporation rate of volatile composition as compared to a corresponding cartridge in which the membrane does not comprise one or more markings obtainable by laser marking.

17. The cartridge according to claim 16, wherein the higher evaporation rate of volatile composition is at least 5% higher when measured over 28 days.

18. A method of making a volatile composition cartridge, comprising:
(i) providing a membrane that has been marked with a laser; and
(ii) manufacturing a volatile composition cartridge that comprises the membrane, wherein the volatile composition cartridge comprises a volatile composition that is configured to evaporate through the membrane.

19. The method according to claim 18, wherein the membrane in step (i) has been marked with a laser using laser charring, laser coloration, or laser dark marking.

20. The method according to claim 18, wherein the membrane in step (i) has been marked with a laser using laser charring.
